(19) United States
(12) Patent Application Publication
(10) Pub. No.: US 2021/0369750 A1
(43) Pub. Date: Dec. 2, 2021

(54) NICOTINAMIDE RIBOSIDE COMPOSITIONS FOR HEALTHSPAN EXTENSION

(71) Applicants: PONCE DE LEON HEALTH DESIGNATED ACTIVITY COMPANY, County Louth (IE); Buck Institute for Research on Aging, Novato, CA (US)

(72) Inventors: David Pereira, Apex, NC (US); Thomas Weldon, Novato, CA (US)

(21) Appl. No.: 17/286,131

(22) PCT Filed: Oct. 23, 2019
(86) PCT No.: PCT/US2019/057684

§ 371 Date: Apr. 16, 2021

Related U.S. Application Data
(60) Provisional application No. 62/750,067, filed on Oct. 24, 2018.

(51) Int. Cl. A61K 31/706; A61K 31/194; A61K 31/09; A61K 9/00; A61P 39/00

(52) U.S. Cl. CPC A61K 31/706; A61K 31/194; A61P 39/00; A61K 9/0053; A61K 31/09

(57) ABSTRACT

Described herein are methods and compositions comprising nicotinamide riboside and alpha-ketoglutarate for treating frailty, for maintaining health, and for re-growing hair.

### CROSS-REFERENCE

This application claims the benefit of U.S. Provisional Application No. 62/750,067 filed Oct. 24, 2018, which is incorporated by reference herein in its entirety.

### SUMMARY OF THE DISCLOSURE

Disclosed herein are methods of delaying onset or delaying progression of frailty in a subject in need thereof comprising administering to the subject a therapeutically effective amount of nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, the delaying onset or delaying progression of frailty comprises delaying onset or delaying progression of a frailty phenotype. In some embodiments, the frailty phenotype is dermatitis. In some embodiments, the frailty phenotype is kyphosis. In some embodiments, the frailty phenotype is tremor. In some embodiments, the frailty phenotype is alopecia. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, the topically administered form is a cream, a foam, a gel, a lotion, an ointment, or a serum.

Disclosed herein are methods for extending healthspan in a subject in need thereof comprising administering to the subject a therapeutically effective amount of a nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, extending healthspan comprises a delay in onset or progression of an age-related phenotype. In some embodiments, the age-related phenotype is selected from the group consisting of graying hair, hair loss, and increased cell senescence. In some embodiments, the age-related phenotype is graying hair. In some embodiments, the age-related phenotype is hair loss. In some embodiments, the age-related phenotype is increased cell senescence. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, the topically administered form is a cream, a foam, a gel, a lotion, an ointment, or a serum.

Disclosed herein are methods for compressing morbidity in a subject in need thereof comprising administering to the subject a therapeutically effective amount of a nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, compressing morbidity comprises a shortening of the time between appearance of a marker of morbidity and end of life. In some embodiments, the marker of morbidity comprises a first heart attack, a first dyspnea from emphysema, a first disability from osteoporosis, or a first memory loss of a certain magnitude. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, the topically administered form is a cream, a foam, a gel, a lotion, an ointment, or a serum.

Disclosed herein are methods for helping to maintain health in a subject in need thereof comprising administering to the subject a therapeutically effective amount of a nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, helping to maintain health comprises helping to maintain a healthy musculoskeletal system. In some embodiments, helping to maintain a healthy musculoskeletal system comprises helping to maintain grip strength. In some embodiments, helping to maintain a healthy musculoskeletal system comprises helping to maintain normal spine curvature. In some embodiments, helping to maintain a healthy musculoskeletal system comprises helping to maintain a normal gait. In some embodiments, helping to maintain a healthy musculoskeletal system comprises helping to maintain normal muscle mass. In some embodiments, helping to maintain health comprises helping to maintain a healthy auditory system. In some embodiments, helping to maintain health comprises helping to maintain a healthy ocular system. In some embodiments, helping to maintain a healthy ocular system comprises helping to maintain normal vision. In some embodiments, helping to maintain health comprises helping to maintain a healthy cardiovascular system. In some embodiments, helping to maintain health comprises helping to maintain a healthy respiratory system. In some embodiments, helping to maintain health comprises helping to maintain a healthy metabolism. In some embodiments, helping to maintain a healthy metabolism comprises helping to maintain a normal body temperature. In some embodiments, helping to maintain a healthy metabolism comprises helping to maintain a healthy body weight. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, the topically administered form is a cream, a foam, a gel, a lotion, an ointment, or a serum.

Disclosed herein are methods for maintain hair density in a subject in need thereof comprising administering to the subject a therapeutically effective amount of a nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, maintaining hair density comprises maintaining a healthy scalp. In some embodiments, maintaining hair density comprises maintaining healthy hair follicles. In some embodiments, maintaining hair density comprises maintaining healthy hair shafts. In some embodiments, maintaining hair density comprises maintaining healthy hair bulbs. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, topically administered form is a cream, a foam, a gel, a lotion, an ointment, a serum, a shampoo, or a conditioner.

Disclosed herein are methods for maintaining hair pigmentation in a subject in need thereof comprising administering to the subject a therapeutically effective amount of a nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, maintaining hair pigmentation comprises helping to maintain a normal level melanin. In some embodiments, the subject has low vitamin B12 levels. In some embodiments, maintaining hair pigmentation comprises maintaining a normal level of melanocyte stem cells. In some embodiments, the therapeutically effective amount of a nicotinamide riboside (NR) and α-ketoglutarate (AKG) is applied to the scalp of the subject. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, the topically administered form is a cream, a foam, a gel, a lotion, an ointment, a serum, a shampoo, or a conditioner.

Disclosed herein are methods for re-growing hair in a subject in need thereof comprising administering to the subject a therapeutically effective amount of a nicotinamide riboside (NR) and α-ketoglutarate (AKG). In some embodiments, AKG is formulated as a calcium salt of AKG. In some embodiments, NR and AKG is administered to the subject as a single composition. In some embodiments, NR and AKG is administered to the subject as separate compositions. In some embodiments, NR and AKG is administered to the subject in a 24 hour period. In some embodiments, pterostilbene is administered to the subject. In some embodiments, the subject has male or female pattern baldness, alopecia areata, telogen effluvium, anagen effluvium, alopecial totalis, alopecia universalis, alopecia barbae, alopecia mucinosa, alopecia traction alopecia, scarring alopecia or trichotillomania. In some embodiments, NR and AKG is applied to the scalp of the subject. In some embodiments, the subject is a mammal. In some embodiments, the mammal is a human. In some embodiments, the mammal is a dog. In some embodiments, the mammal is a cat. In some embodiments, the mammal is livestock. In some embodiments, the livestock is selected from the group consisting of cattle, sheep, goats, swine, poultry, and equine animals. In some embodiments, the NR and AKG are administered once daily. In some embodiments, the NR and AKG are administered twice daily. In some embodiments, the NR and AKG are administered in the morning and evening. In some embodiments, the NR and AKG are administered once a week. In some embodiments, the NR and AKG are administered once a month. In some embodiments, the NR and AKG are formulated into an orally administered form. In some embodiments, the orally administered form comprises a tablet, a powder, a suspension, a serum, an emulsion, a capsule, a granule, a pill, a gel, a solution, or a syrup. In some embodiments, the orally administered form is a sustained release dosage form. In some embodiments, the orally administered form is formulated into animal feed. In some embodiments, the orally administered form is the gel. In some embodiments, the NR and AKG are formulated into a topically administered form. In some embodiments, the topically administered form is a cream, a foam, a gel, a lotion, an ointment, a serum, a shampoo, or a conditioner.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### Certain Terminology

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In certain embodiments, the term “delay” or “delaying” as related to a disease or disorder may refer to a compound that, in a statistical sample, delays or postpones the occurrence of the disorder or condition in the treated sample relative to an untreated control sample, or delays the onset or reduces the severity of one or more symptoms of the disorder or condition relative to the untreated control sample.

As used herein, “α-ketoglutarate” or AKG comprises derivatives of α-ketoglutarate (e.g., the derivatives set forth in MacKenzie, et al. (2007) Mol Cell Biol 27(9):3282-3289)), analogues of α-ketoglutarate (e.g., phosphonate analogues (e.g., those recited in Bunik, et al. (2005) Biochemistry 44(31):10552-61), esters of α-ketoglutarate (e.g., dimethyl α-ketoglutarate and octyl α-ketoglutarate), and various species specific analogues, e.g., human α-ketoglutarate, porcine α-ketoglutarate, murine α-ketoglutarate, bovine α-ketoglutarate, and the like.

### Compositions

Described herein are methods and compositions for pharmacological treatment of lifespan, healthspan, and aging-related disease. Further disclosed herein, in some aspects, are methods and compositions for delaying onset, or delaying progression of a disorder, reversing an age-related phenotype, extending healthspan, compressing morbidity, and reducing formation of senescent cells. In some embodiments, are methods and compositions for treating frailty, or for re-growing hair.

In some embodiments, are methods and compositions for helping to maintain health. In some embodiments, are methods and compositions for maintaining hair density. In some embodiments, are methods and compositions for maintaining hair pigmentation.

In some embodiments, the compositions disclosed herein comprise alpha-ketoglutarate (AKG). In some embodiments, α-ketoglutarate (Formula 1) is also known as 2-oxopentanedioic acid, 2-ketoglutaric acid, 2-oxoglutaric acid, and oxoglutaric acid. At physiological pH, α-ketoglutarate exists in the deprotonated form depicted as Formula 2. α-ketoglutarate is an intermediate in the Krebs cycle of eukaryotic organisms and is biosynthesized from isocitrate (in the Krebs cycle process) or L-glutamate (via alanine transaminase) in such organisms. Both α-ketoglutarate and its corresponding salts are commercially available, either via preparation from fermentation cultures (for example see U.S. Pat. No. 2,776,926) or chemical synthesis from closely related compounds.

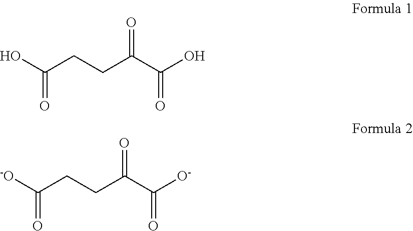

Consistent with its role in energy generation via the Krebs cycle, α-ketoglutarate is an important regulator of bioenergetics in cells and is implicated as an inhibitor of ATP synthase subunit β and an indirect inhibitor of the kinase mTOR, a consequence of partial inhibition of the mitochondrial electron transport chain.

In some embodiments, α-ketoglutarate is provided as the free acid (α-ketoglutaric acid). In other embodiments, α-ketoglutarate is provided as a monosodium salt, a disodium salt, or a monopotassium salt. In further embodiments, disodium salts of α-ketoglutarate are provided as anhydrous salts, monohydrates, or dihydrates. In yet further embodiments, α-ketoglutarate is provided as a mono- or di-valent salt with other cations described in the U.S. FDA Orange Book. Such cations include calcium, diolamine, lithium, lysine, magnesium, meglumine, olamine, tromethamine, and zinc.

Further disclosed herein, in certain aspects, are compositions that comprise α-ketoglutarate salt. In some embodiments, α-ketoglutarate is provided as a calcium salt (Ca-AKG). In some embodiments, calcium α-ketoglutarate can be a hydrate calcium α-ketoglutarate. In some embodiments, calcium α-ketoglutarate can be a mono-hydrate calcium α-ketoglutarate. In some embodiments, calcium α-ketoglutarate can be hemi-hydrate calcium α-ketoglutarate. In some embodiments, calcium α-ketoglutarate can be anhydrous calcium α-ketoglutarate.

In some embodiments, the compositions disclosed herein comprise an ester of α-ketoglutarate. In some embodiments, the ester of α-ketoglutarate is a methyl ester of α-ketoglutarate. In some embodiments, the ester of α-ketoglutarate is a dimethyl ester of α-ketoglutarate. In some embodiments, the ester of α-ketoglutarate is an ethyl ester of α-ketoglutarate. In some embodiments, the ester of α-ketoglutarate is a diethyl ester of α-ketoglutarate.

In some embodiments, AKG or Ca-AKG is combined with fish oil. In some embodiments, AKG or Ca-AKG is formulated with essential amino acids, including one or more of L-histidine, L-isoleucine, L-leucine, L-lysine, L-methionine, L-phenylalanine, L-threonine, or L-valine. In some embodiments, Ca-AKG or AKG is combined with nordihydroguaiaretic acid.

In some embodiments, the compositions disclosed herein comprise nicotinamide riboside (NR). In some embodiments, NR (Formula 3) is also known as 1-(β-D-ribofuranosyl)nicotinamide or N-ribosylnicotinamide. As used herein, “nicotinamide riboside” or “NR” also includes nicotinamide riboside salts, such as nicotinamide riboside chloride.

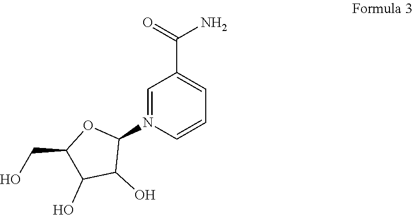

In some embodiments, the compositions disclosed herein comprise pterostilbene or an analog of polyphenol reservatrol. In some embodiments, pterostilbene (Formula 4) is also known as 4-[(E)-2-(3,5-dimethoxyphenyl)ethenyl]phenol, 3′,5′-dimethoxy-4-stilbenol, 3,5-Dimethoxy-4′-hydroxy-E-stilbene, or 3′,5′-dimethoxy-resveratrol. As used herein, “pterostilbene” also includes salts of pterostilbene.

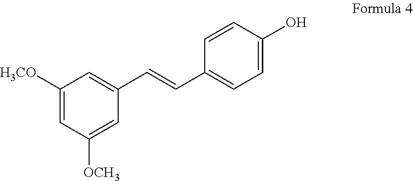 [SMILES: COc1cc(/C=C/c2ccc(O)cc2)cc(OC)c1]

In some embodiments, the compositions disclosed herein comprise AKG and NR. In some embodiments, the compositions disclosed herein comprise Ca-AKG and NR. In some embodiments, the compositions disclosed herein comprise AKG, NR, and pterostilbene. In some embodiments, the compositions disclosed herein comprise Ca-AKG, NR, and pterostilbene.

In some embodiments, AKG and NR are administered in combination but as separate compositions. In some embodiments, Ca-AKG and NR are administered in combination but as separate compositions. In some embodiments, AKG, NR, and pterostilbene are administered in combination but as separate compositions. In some embodiments, Ca-AKG, NR, and pterostilbene are administered in combination but as separate compositions.

### Methods of Use

### Frailty

In an aspect, the disclosure provides methods for treating, delaying onset, or delaying progression of frailty using the active agents or compositions thereof described herein. The term “frailty” refers to a biological syndrome of decreased reserve and resistance to stressors due to decline in multiple physiological systems. Subjects suffering from frailty have improved likelihood of adverse health outcomes to events that stress one or more of their physiological systems. In humans, frailty frequently presents via non-specific symptoms, falls, delirium, fluctuating disability, or a combination thereof. Non-specific symptoms include extreme fatigue, unexplained weight loss, and frequent infections. Falls include hot falls (minor illness reducing postural balance below a threshold to maintain stability) or spontaneous falls (vital postural systems declining as a result of declines in vision, balance, and strength). Delirium refers to rapid onset of fluctuating confusion and impaired awareness. Fluctuating disability refers to day-to-day instability in the ability of a patient to function independently.

Various clinical scoring and evaluation systems for frailty are known to those skilled in the art and are suitable for assessing the effects of treatment on frailty. In some embodiments, frailty is evaluated in humans using the 70-item CSHA Frailty Index (see, for e.g. Theou et al. Age Ageing 42: 614-619 (2013)). A brief description of how the index is employed follows:

Items including the presence and/or severity of current diseases, ability in daily living and physical signs from the clinical and neurological examinations (see items in Table 1 below) are evaluated. Each deficit is dichotomized or trichotomized and mapped to the interval 0-1 (i.e. individual items had scores of 0, 0.33, 0.50, 0.67 or 1.0), representing the occurrence and severity of the problem. For each person, a 70-dimensional vector is constructed, such that a person with 5 deficits would have a score of 5/70=0.071.

TABLE 1
List of measures used to construct the Frailty Index

| | | Limitations with |
|---|---|---|
| Self-rated health | Long-term illness | Activities |
| In hospital last 12 | Heart attack | High blood pressure |
| months | | |
| High blood | Stroke or cerebral | Diabetes or high blood |
| cholesterol | vascular Disease | sugar |
| Chronic lung | Asthma | Arthritis |
| disease | | |
| Osteoporosis | Cancer | Stomach or duodenal |
| | | ulcer |
| Parkinson disease | Cataracts | Hip or femoral fracture |
| Pain in any joint | Heart trouble or angina | Breathlessness |
| Persistent cough | Swollen legs | Sleeping problems |
| Falling down | Fear of falling down | Dizziness |
| Stomach or intestine | Incontinence | Dentures |
| Problems | | |
| Biting on hard foods | Eyesight | Hearing |
| Walking 100 meters | Sitting for about two | Getting up from a chair |
| | hours | after prolonged sitting |
| Climbing several | Stooping, kneeling, or | Reaching or extending |
| flights of Stairs | Crouching | arm above shoulder |
| | | level |
| Pulling or pushing | Lifting or carrying | Picking up a small coin |
| large Objects | weights over 10 | from a table |
| | pounds/5 kilos | |
| Dressing | Walking across a room | Bathing or showering |
| Eating | Getting in or out of | Using the toilet |
| | bed | |
| Using a map to figure | Preparing a hot meal | Shopping for groceries |
| out how to get around | | |
| Making telephone | Taking medications | Doing work around the |
| calls | | house or garden |
| Managing money | Vigorous activities | Moderate Activities |
| Orientation | Mathematical | Verbal fluency score |
| | performance | |
| Delayed recall test | Depression | Pessimism |
| Suicidality | Sleep | Interest |
| Appetite | Fatigue | Concentration |

Further detail on application of the CSHA frailty index (e.g. calculation of scores for individual measures) can be found in other publications in the field, for e.g. in Searle et al. A standard procedure for creating a frailty index. BMC Geriatrics 8:24 (2008). An example of the use of the CSHA frailty index in humans can be found in Example 4, where selection of pre-frail individuals for treatment and evaluation of pharmacological treatment of frailty is described.

In other embodiments, frailty is evaluated in non-human animals, such as mice. Recognized signs of frailty in mice correspond to many of those in humans, and involve metabolic (e.g. body temperature, body weight), integumental (e.g. alopecia, loss of fur color, dermatitis, loss of whiskers, grooming), physical/musculoskeletal (e.g. tumors, distended abdomen, kyphosis, tail stiffening, gait disorder, tremor, decreased forelimb grip strength, body condition/muscle wasting/obesity), vestibulocochlear/auditory (e.g. vestibular disturbance, hearing loss), ocular/nasal (e.g. cataracts, corneal opacity, eye discharge, microphthalmia, vision loss, increased menace reflex, nasal discharge), digestive/urogenital (e.g. malocclusions, rectal prolapse, vaginal/uterine/penile prolapse, diarrhea), respiratory (e.g. abnormal breathing rate or depth), and discomfort symptoms (e.g. increased mouse grimace scale, piloerection).

In some embodiments, frailty is assessed in mice via a 31-item clinical frailty index encompassing the 31 example phenotypes recited above as described in, for e.g. Whitehead et al. J Gerontol A Biol Sci Med Sci 69:621-632 (2014). Clinical examinations are performed at approximately the same time every 2 to 3 months, and involve body weight and surface temperature measurement by abdominal infrared, followed by a clinical exam to evaluate the 31 frailty phenotypes. The severity of each deficit is rated on a scale, with 0 given for no sign of a deficit, 0.5 for a mild deficit, and 1 for a severe deficit. Deficits in body weight (g) and body surface temperature (° C.) are scored in quantiles between 0 and 1 based on number of standard deviations from reference values in young adult animals (0.25, 0.5, 0.75, and 1.0) according to how many standard deviations the score varies from the mean (1 is >3SD). The sum of the scores for each parameter produce the final 31-item frailty index, which can be compared between individual mice according to standard statistical techniques to assess frailty.

In some embodiments, frailty is assessed in mice via an abbreviated with an eight-item functional frailty index as described in, for e.g. Whitehead et al. J Gerontol A Biol Sci Med Sci 69:621-632 (2014) and Parks et al. J Gerontol A Biol Sci Med Sci. 67:217-227 (2012). In this method, 7 performance parameters based on open-field behavior of mouse subjects are assessed: 1) total distance moved in 10 minutes; 2) maximal distance moved between bouts of inactivity; 3) total duration of movement (seconds); 4) percent of total time spent moving; 5) the change in direction per unit distance moved, called meander (degrees/cm; from 0° to 180°); 6) the average velocity of movement over 10 minutes (cm/s); and 7) rearing frequency (number of occurrences/10 min). An eighth non-movement parameter, weight, is additionally assessed. Open-field assessments are performed between 10 am and noon each day. Mice are weighed and activity was recorded with automated video tracking software for 10 minutes in an open-field arena. Videos are digitized with an analog-to-digital converter and analyzed with video tracking analysis software to obtain values for the parameters used to create the eight-item frailty index. Mean and standard deviation for each of these parameters are calculated and assigned to a score quantile between 0 and 1 (0.25, 0.5, 0.75, and 1.0) according to how many standard deviations the score varies from the mean (1 is >3SD). The parameters are added, and divided by eight to receive a frailty index score between 0 and 1 for the mouse subjects. Higher scores correspond to increasingly frail mice.

In some embodiments, the disclosure provides methods for treating, delaying onset, or delaying progression of frailty in a subject in need thereof using the compositions disclosed herein. In some aspects, the composition for treating, delaying onset, or delaying progression of frailty comprises two or more active agents.

In some embodiments, treating, delaying onset, or delaying progression of frailty in a subject in need thereof comprises administering to the subject AKG and NR. In some embodiments, treating, delaying onset, or delaying progression of frailty in a subject in need thereof comprises administering to the subject Ca-AKG and NR. In some embodiments, treating, delaying onset, or delaying progression of frailty in a subject in need thereof comprises administering to the subject AKG, NR, and pterostilbene. In some embodiments, treating, delaying onset, or delaying progression of frailty in a subject in need thereof comprises administering to the subject Ca-AKG, NR, and pterostilbene.

### Healthspan

In an aspect, the disclosure provides methods of treatment for extending healthspan using the active agents or compositions thereof disclosed herein. Healthspan refers to the period of time during which an individual meets one or more selected measures of health. An increase in healthspan refers to an extension in the period of health, according to such measures, as compared to the period of health in a control population. Examples of selected measures of health that are evaluated in a human population to assess healthspan include one or more age-related phenotypes such as energetics/metabolism (e.g. elevated insulin, insulin resistance, elevated fasting blood glucose+GTT, elevated Hb A1c, adiponectin, elevated DEXA/abdominal adiposity, increased IGF-I, decreased T3, elevated low-density lipoprotein, decreased high-density lipoprotein, elevated triglycerides), skeletal muscle function (e.g. decreased hand grip strength, decreased mobility), cardiopulmonary function (e.g. decreased VO2max, elevated blood pressure, decreased pulse wave velocity, intima media thickness, decreased left ventricular diastolic function, increased left ventricular diastolic pressure), inflammation and immune function (e.g. decreased lymphocyte number, decreased lymphoid/myeloid ratio, elevated CRP, elevated IL-6, elevated TNF-α), sensory function (e.g. decreased visual acuity, decreased nerve conduction velocity), cognition (e.g. decreased score on cognitive function tests like the MMSE/AMTS/GPAC, impaired activity via (MRIs), cellular senescence (e.g. graying hair), and pathology (e.g. renal, cardiac, pulmonary, breast, or prostate tissue evaluation to evaluate age-related tissue hypertrophy or dysplasia).

Examples of non-human animals used for assessment of lifespan-extension or healthspan interventions include C. elegans, D. melanogaster, and M. musculus. Use of D. melanogaster or M. musculus to evaluate lifespan or healthspan interventions are found in, for e.g. Bauer et al. Proc Natl Acad Sci USA. 101:12980-5 (2004) and Selman et al. FASEB J 22:807-18 (2008).

In some embodiments, the disclosure provides methods of extending healthspan using the compositions disclosed herein. In some aspects, the composition comprises two or more active agents described herein.

In some embodiments, extending healthspan in a subject in need thereof comprises administering to the subject AKG and NR. In some embodiments, extending healthspan in a subject in need thereof comprises administering to the subject Ca-AKG and NR. In some embodiments, extending healthspan in a subject in need thereof comprises administering to the subject AKG, NR, and pterostilbene. In some embodiments, extending healthspan in a subject in need thereof comprises administering to the subject Ca-AKG, NR, and pterostilbene.

### Compressing of Morbidity

In an aspect, the disclosure provides methods of compressing morbidity using the active agents or compositions thereof described herein. Compression of morbidity occurs if the age of first appearance of aging manifestations and chronic disease symptoms increases more rapidly than life expectancy. The period between marker of morbidity (e.g. first heart attack, first dyspnea from emphysema, first disability from osteoarthritis, first memory loss of a certain magnitude) and the end of life is shortened when the average onset age of the marker increases more rapidly than life expectancy from the same age. This disproportionally increases the healthy years of life, and dramatically reduces the end stage costs of healthcare.

In an aspect, the disclosure provides methods of compressing morbidity using the active agents or compositions thereof described herein. In some aspects, the composition comprises two or more active agents as described herein.

In some embodiments, compressing morbidity in a subject in need thereof comprises administering to the subject AKG and NR. In some embodiments, compressing morbidity in a subject in need thereof comprises administering to the subject Ca-AKG and NR. In some embodiments, compressing morbidity in a subject in need thereof comprises administering to the subject AKG, NR, and pterostilbene. In some embodiments, compressing morbidity in a subject in need thereof comprises administering to the subject Ca-AKG, NR, and pterostilbene.

### Methods of Helping to Maintaining Health

In an aspect, the disclosure provides methods of helping to maintain health using the active agents or compositions thereof described herein. In some embodiments, helping to maintain health comprises helping to maintain a healthy metabolism. In some embodiments, helping to maintain a healthy metabolism comprises helping to maintain a healthy body temperature. In some embodiments, helping to maintain a healthy metabolism comprises helping to maintain a healthy body weight.

In some embodiments, helping to maintain health comprises helping to maintain a healthy musculoskeletal system. In some embodiments, helping to maintain a healthy musculoskeletal system comprises helping to maintain grip strength. In some embodiments, helping to maintain a healthy musculoskeletal system comprises helping to maintain normal spine curvature. In some embodiments, helping to maintain a healthy musculoskeletal system comprises helping to maintain normal gait. In some embodiments, helping to maintain a healthy musculoskeletal system comprises helping to maintain normal muscle mass.

In some embodiments, helping to maintain health comprises helping to maintain a healthy auditory system. In some embodiments, helping to maintain health comprises helping to maintain a healthy ocular system. In some embodiments, helping to maintain health comprises helping to maintain normal vision. In some embodiments, helping to maintain health comprises helping to maintain a healthy digestive system. In some embodiments, helping to maintain health comprises helping to maintain a healthy urogenital tract. In some embodiments, helping to maintain health comprises helping to maintain a healthy respiratory system.

In some embodiments, helping to maintain health comprises helping to maintain a healthy cardiovascular system. In some embodiments, helping to maintain health comprises helping to maintain a healthy body weight.

In some embodiments, the disclosure provides methods of helping to maintain health comprise using the compositions disclosed herein. In some aspects, the composition comprises two or more active agents as described herein.

In some embodiments, helping to maintain health in a subject in need thereof comprises administering to the subject AKG and NR. In some embodiments, helping to maintain health in a subject in need thereof comprises administering to the subject Ca-AKG and NW In some embodiments, helping to maintain health in a subject in need thereof comprises administering to the subject AKG, NR, and pterostilbene. In some embodiments, helping to maintain health in a subject in need thereof comprises administering to the subject Ca-AKG, NR, and pterostilbene.

### Maintaining Hair Density

In an aspect, the disclosure provides methods of maintaining hair density using the active agents or compositions thereof described herein. In some embodiments, hair density is maintained in a subject that has alopecia. In some embodiments, maintaining hair density comprises maintaining a healthy scalp. In some embodiments, maintaining hair density comprises maintaining healthy hair follicles. In some embodiments, maintaining hair density comprises maintaining healthy hair shafts. In some embodiments, maintaining hair density comprises maintaining healthy hair bulbs.

In some embodiments, the disclosure provides methods of maintaining hair density comprise using the compositions disclosed herein. In some aspects, the composition comprises two or more active agents as described herein.

In some embodiments, maintaining hair density in a subject in need thereof comprises administering to the subject AKG and NR. In some embodiments, maintaining hair density in a subject in need thereof comprises administering to the subject Ca-AKG and NR. In some embodiments, maintaining hair density in a subject in need thereof comprises administering to the subject AKG, NR, and pterostilbene. In some embodiments, maintaining hair density in a subject in need thereof comprises administering to the subject Ca-AKG, NR, and pterostilbene.

### Maintaining Hair Pigmentation

In an aspect, the disclosure provides methods of maintaining hair pigmentation using the active agents or compositions thereof described herein. In some embodiments, maintaining hair pigmentation comprises maintaining a normal level of melanin. In some embodiments, hair pigmentation is maintained in a subject that has low vitamin B12 levels. In some embodiments, maintaining hair pigmentation comprises maintaining a normal level of melanocyte stem cells.

In some embodiments, the disclosure provides methods of maintaining hair pigmentation comprises using the compositions disclosed herein. In some aspects, the composition comprises two or more active agents as described herein.

In some embodiments, maintaining hair pigmentation in a subject in need thereof comprises administering to the subject AKG and NR. In some embodiments maintaining hair pigmentation in a subject in need thereof comprises administering to the subject Ca-AKG and NR. In some embodiments, maintaining hair pigmentation in a subject in need thereof comprises administering to the subject AKG, NR, and pterostilbene. In some embodiments, maintaining hair pigmentation in a subject in need thereof comprises administering to the subject Ca-AKG, NR, and pterostilbene.

### Hair Re-Growth

In some embodiments, the disclosure provides methods of re-growing hair in a subject in need thereof using the active agents or compositions thereof described herein. In some embodiments, the subject has alopecia. In some embodiments, the subject has male or female pattern baldness. In some embodiments, the subject has alopecia areata. In some embodiments, the subject has telogen effluvium. In some embodiments, the subject has anagen effluvium. In some embodiments, the subject has alopecial totalis. In some embodiments, the subject has alopecia universalis. In some embodiments, the subject has alopecia barbae. In some embodiments, the subject has alopecia mucinosa. In some embodiments, the subject has traction alopecia. In some embodiments, the subject has scarring alopecia. In some embodiments, the subject has trichotillomania.

In some embodiments, re-growing hair in a subject in need thereof comprises administering to the subject AKG and NR. In some embodiments, re-growing hair in a subject in need thereof comprises administering to the subject Ca-AKG and NR. In some embodiments, re-growing hair in a subject in need thereof comprises administering to the subject AKG, NR, and pterostilbene. In some embodiments, re-growing hair in a subject in need thereof comprises administering to the subject Ca-AKG, NR, and pterostilbene.

### Routes of Administration

The compositions according to the disclosure herein may be administered via a variety of routes. In some embodiments, the composition is formulated for oral administration. In some embodiments, the composition is formulated for sublingual administration. In other embodiments, the composition is formulated for injection. In some embodiments, the composition is formulated for topical administration.

In some embodiments, the compounds described herein are formulated in oral dosage forms. Two or more compounds according to the invention are formulated by combining them with, e.g., pharmaceutically acceptable carriers or excipients. In various embodiments the compounds according to the invention are formulated in oral dosage forms including, by way of example only, tablets, powders, granules, pills, dragees, capsules, liquids, serums, gels, solutions, syrups, elixirs, slurries, suspensions, emulsions and the like.

### Orally Administered Forms

In certain embodiments, preparations containing the active agents for oral use are obtained by mixing one or more solid excipient with one or more of the compounds described herein, optionally grinding the resulting mixture, and processing the mixture of granules, after adding suitable auxiliaries, if desired, to obtain tablets, pills, or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, dosage forms, such as dragee cores, pills, and tablets, are provided with one or more suitable coatings. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of the active agents described herein are formulated into other solid oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added. In some embodiments, AKG or Ca-AKG is formulated into a soft gel capsule.

In some embodiments, AKG or Ca-AKG are formulated as coated beads as described in Patel, R R and Patel J K, “Novel Technologies of Oral Controlled Release Drug Delivery System,” Systematic Reviews in Pharmacy, July-December 2010, Vol. 1 (2), 128-132.

In some embodiments, AKG is formulated with fish oil in a gel cap. In some embodiments, AKG or Ca-AKG is formulated into an amino acid supplement including one or more of the following amino acids, L-histidine, L-isoleucine, L-leucine, L-lysine, L-methionine, L-phenylalanine, L-threonine, L-valine. In some embodiments, AKG or Ca-AKG is added as a source of glutamic acid.

In other embodiments, active agents described herein are formulated into oral liquid dosage forms. Exemplary liquid preparations for oral use include solutions, emulsions, serums, solutions, syrups or suspensions containing one or more active ingredients in a suitable vehicle. Syrups are clear viscous oral liquids containing high concentrations of sugar or other sweetening agents, in which active agents are solubilized in a pharmaceutically acceptable vehicle. Suspensions consist of finely divided particles of active agent suspended in pharmaceutically acceptable vehicle in which the particles are poorly soluble. Oral emulsions contain liquid forms of active agents dispersed as droplets in a continuous phase of another immiscible vehicle with the help of emulsifying agents (e.g. carbohydrates, gelatin, high molecular weight alcohols, wetting agents, colloidal clays, and the like).

In some embodiments, active agents are formulated into semi-solid oral dosage forms such as gels. Gels or jelly-like formulations have particular relevance for elderly or dysphagic patients with difficulty consuming other oral dosage forms. Gels are formed by adding active agents to water, adding a low critical concentration (e.g. 0.5-2.5%) of a gelling agent, heating, and cooling. Examples of suitable gelling agents include agar, gelatin, carrageenan, sodium caseinate, glycerogelatin, silk fibroin, gellan gum, kelcogel, xyloglucan, gellan, and pectin.

### Animal Feed

In certain embodiments, the active agents are included in a diet which can comprise any suitable pet food formulation which also provides adequate nutrition for a non-human animal. For example, a typical canine diet for use in the present invention may contain about 18-40% crude protein, about 4-30% fat, and about 4-20% total dietary fiber. However, no specific ratios or percentages of these or other nutrients are required. Examples of detailed preparation of animal feed from base ingredients are found elsewhere, for e.g. in U.S. Pat. No. 4,045,585, US20100303968, and U.S. Pat. No. 3,875,304.

### Altered Release

A composition comprising any of the active agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of agent contained within a sustained release formulation depends upon the site of implantation, the rate and expected duration of release, and the nature of the condition, disease or disorder to be treated or prevented.

### Injectable

In still other embodiments, the active agents described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, formulations for parenteral administration include aqueous solutions of the active compounds in water-soluble form. In additional embodiments, suspensions of the active agents are prepared as appropriate oily injection suspensions. Suitable lipophilic solvents or vehicles for use in the compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

### Topical

In still other embodiments, the active agents are administered topically. The compounds described herein are formulated into a variety of topically administrable compositions, such as solutions, suspensions, lotions, gels, foams, serums, pastes, medicated sticks, balms, creams or ointments. Such compositions optionally contain solubilizers, stabilizers, tonicity enhancing agents, buffers and preservatives.

Compositions disclosed herein can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

In certain embodiments, the compositions comprising the active agents are formulated for topical administration using a bandage or transdermal patch, or as a powder/talc or other solid, liquid, spray, aerosol, ointment, foam, cream, gel, or paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

### Hair Care Products

In some embodiments, the active agents according to the invention are formulated into a hair care product. In some embodiments, the active agent in the hair care product comprises alpha-ketoglutarate salt. Examples of hair care products useful for administering the two or more compounds include a shampoo, a conditioner, a hair spray, or a moisturizer. In some embodiments, the alpha-ketoglutarate is formulated in a shampoo. A shampoo is a preparation comprising a surfactant (for e.g. sodium lauryl sulfate) and other additives specifically selected to remove surface grease, dirt, and skin debris from the hair shaft and scalp. An exemplary liquid shampoo formulation would be an aqueous solution containing 40% sodium lauryl sulfate, 2-4% sodium chloride (adjusted to desired viscosity), an effective amount of the 2 or more compounds, a preservative, and optional perfumes or colors. A conditioner or moisturizer is a preparation comprising a conditioning or moisturizing substance which adheres to hair in the presence of water. Examples of conditioning or moisturizing substances suitable for use in conditioners include quaternized surfactants, cationic polymers, silicone compounds (for e.g. polydimethylsiloxane, cyclomethicone), emollients, and humectants. An exemplary hair spray comprises a near 50:50 mix of buffered water and ethanol as diluents, alongside low concentrations of a humectant (e.g. glycerol), a conditioner, and a hair styling polymer (e.g. PVP K-30).

### Administration Protocol and Schedule

In some embodiments one or more of the active agents are administered separately. In some embodiments, the active agents administered separately are administered in separate dosage units (e.g. pills, dragees, tablets). In some embodiments, the active agents administered separately are administered via separate routes of administration. In certain embodiments, two or more of the active agents are administered separately. In certain embodiments, three of the active agents are administered separately. In certain embodiments, the active agents administered separately are not administered simultaneously.

In certain embodiments, the active agents not administered simultaneously are administered within a defined window. In specific embodiments, the defined window is 48, 36, 24, 12, or 6 hours. In some embodiments one or more, two or more, or three of the active agents are administered within the defined window.

In some embodiments, the active agents or compositions thereof used for treatment are administered for a particular treatment period. In some embodiments, the active agents or compositions thereof are administered for a chronic treatment period, that is, for an extended period of time, including throughout the duration of the patient's life in order to ameliorate or otherwise control or limit the symptoms of the patient's disease or condition. Within the treatment period, the active agents or compositions thereof used for treatment are administered on a particular time schedule. In further embodiments, the active agents or compositions thereof are administered one, two, three, or four times daily. In some embodiments, the active agents or compositions thereof are administered in the morning and evening. In some embodiments, the active agents or compositions thereof are administered one, two, three, or four times weekly. In some embodiments, the active agents or compositions thereof are administered one, two, three, or four times monthly. In some embodiments the active agents or compositions are taken for at least three months with one year. In some embodiments, the active agents or compositions are taken one month of every six months.

### Examples

### Example 1. Active Agent Trial in a Frailty Mouse Model

### Animals

C57BL/6J mice of both sexes are aged in group caging in an approved animal facility. Mice are maintained on a 12-hour light/dark cycle, with free access to food and water. In general, initiation of interventions occurs in mice after 12 months of age and is maintained throughout the lifespan of the mice. However, short term or periodic interventional strategies may also be tested.

Intervention and Assessment

Mice are randomly assigned to either treatment or control group, and administered compositions of active agents or placebo (control) in food or water for a sufficient treatment period to observe effects on frailty characteristics.

Mice are periodically evaluated on the 31-item clinical frailty index previously described herein. This was first reported by Whitehead et al. (Journals of Gerontology: BIOLOGICAL SCIENCES, 69:621-632; 2014). This has been used in studies recently as a valuable metric of mouse frailty that involves non-invasive scoring ideal for longitudinal studies and corresponds well with the onset of human frailty (Rockwood et al., Scientific Reports, 7, 43068, 2017).

The 31 point metric for assessment of animal frailty is included in the following table:

| | | |
|---|---|---|
| Alopecia | Tumors | Diarrhea |
| Coat Condition | Vestibulocochlear/ | Malocclusions |
| | Auditory | |
| Dermatitis | Hearing Loss | Rectal prolapse |
| Loss of Fur Color | Vestibular disturbance | Vaginal/uterine/ |
| | | penile prolapse |
| Loss of whiskers | Ocular/Nasal | Respiratory |
| Physical/Musculoskeletal | Cataracts | Breathing rate/depth |
| Body condition score | Corneal discharge | Discomfort |
| Distended abdomen | Eye discharge/swelling | Mouse grimace scale |
| Gait disorder | Menace reflect | Piloerection |
| Grip strength | Microphthalmia | Other |
| Kyphosis | Nasal discharge | Temperature |
| Tail stiffening | Vision loss | Weight |

For all individual frailty metrics shown, a similar strategy is used to assess frailty score. Using a double blind method, scorers are asked to assess the relative score of each animal either as 0 (healthy), 0.5 (moderately unhealthy) and 1 (unhealthy). For total frailty, all 31 metrics are added and divided by 31.

To make all scoring consistent, each scorer is periodically asked to assess the same animal and any differences in scores are mediated and resolved.

Loss of fur color and appearance of grey hair is used interchangeably.

Alopecia or hair loss is used interchangeably.

Kyphosis is a measure of exaggerated outward curvature of the lower cervical/thoracic vertebral column, and can reflect either loss of bone density or increased muscle weakness.

Tremors are a measure of involuntary shaking at rest or during movement.

Dermatitis is a routine occurrence in aging C57/B16 mice.

Body condition score involves visual scoring and/or palpitation to assess the state of health of the animal. Signs of frailness result in higher frailty scores.

Piloerection is defined as involuntary hair bristling and can be thought of similarly to “goose bumps.” This increases with age and can be triggered by coldness, reflecting an inability of the mice to maintain body temperature.

### Example 2. Cell Senescence Assay

To measure the effects of active agents on cell senescence, IMR-90 fibroblasts in culture are exposed to gamma-irradiation either after pretreatment with active agent or prior to (post) treatment with an active agent. Cell senescence is measured by β-Galactosidase fluorescence. Pretreatment of active agents which protect cells from irradiation-induced senescence, suggests that one mechanism by which active agents might mediate lifespan extension and frailty reduction is through prevention of cell senescence.

### Example 3. Testing of Calcium Alpha-Ketoglutarate (CaAKG) and Nicotinamide Riboside (NR) in a Frailty Mouse Model

### Animals

C57BL/6J female mice are aged in group caging in an approved animal facility. Mice are maintained on a 12-hour light/dark cycle, with free access to food and water.

Intervention and Assessment

Mice are divided into four groups with each group containing 25 mice: (1) control, (2) CaAKG, (3) NR, and (4) CaAKG and NR combination.

The CaAKG is milled into the rodent diet and contains 18% protein. The rodent diet is purchased from Envigo, catalog number 2918 (irradiated) https://www.envigo.com/products-services/teklad/laboratory-animal-diets/natural-ingredient/rodent/2018-diets.aspx.

The NR is delivered to the mice in a 0.3% NR in water solution.

Baseline assessment is assessed at 16 months old mice. Treatment begins when mice reach 17 months old. Frailty index is assessed in every 2 months after treatment according to the 31-item clinical frailty index as described in Example 1. Blood collection, hemodynamic, behavioural assessment and urine collection is done at 3 months and 6 months after treatment. Metabolic biomarker screening with Nightingale blood test is done at 10 months after treatment. Life span of the animals is monitored every day and life expectancy will be recorded. Histology of various tissues will be performed post mortem to study the cause of death of each mouse and toxicology of compounds.

A timeline for the study is shown in Table 2.

TABLE 2
Timeline of study

| Mice | | | | | | | | | | |
|---|---|---|---|---|---|---|---|---|---|---|
| age | | Frailty | | Cytokines- | | | Hemo- | | Night- | |
| (mo) | Treatment | index¹ | Rotarod² | Multiplex³ | CBC³ | FACs³ | dynamics⁴ | Urine⁵ | ingale¹ | Methylation¹ |
| 15 | | | | | | | | | | |
| 16 | | Week 1 | Week 1 | Week 2 | Week 2 | Week 2 | Week 2 | Week 2 | | |
| 17 | Month 0 | | | | | | | | | |
| 18 | Month 1 | | | | | | | | | |
| 19 | Month 2 | Week 1 | | | | | | | | |
| 20 | Month 3 | | Week 1 | Week 2 | Week 2 | Week 2 | Week 2 | Week 2 | | |
| 21 | Month 4 | Week 1 | | | | | | | | |
| 22 | Month 5 | | | | | | | | | |
| 23 | Month 6 | Week 1 | Week 1 | Week 2 | Week 2 | Week 2 | Week 2 | Week 2 | | |
| 24 | Month 7 | | | | | | | | | |
| 25 | Month 8 | Week 1 | | | | | | | | |
| 26 | Month 9 | | | | | | | | | |
| 27 | Month 10 | Week 1 | | | | | | | Week 1 | Week 1 |
| 28 | Month 11 | | | | | | | | | |
| 29 | Month 12 | Week 1 | | | | | | | | |
| 30 | Month 13 | | | | | | | | | |
| 31 | Month 14 | Week 1 | | | | | | | | |
| 32 | Month 15 | | | | | | | | | |
| 33 | Month 16 | Week 1 | | | | | | | | |
| 34 | Month 17 | | | | | | | | | |
| 35 | Month 18 | Week 1 | | | | | | | | |
| 36 | Month 19 | | | | | | | | | |
| 37 | Month 20 | Week 1 | | | | | | | | |
| 38 | Month 21 | | | | | | | | | |
| 39 | Month 22 | Week 1 | | | | | | | | |
| 40 | Month 23 | | | | | | | | | |
| 41 | Month 24 | Week 1 | | | | | | | | |
| 42 | Month 25 | | | | | | | | | |
| 43 | Month 26 | Week 1 | | | | | | | | |
| 44 | Month 27 | | | | | | | | | |

¹Phenotype *Frailty index 300 mice take 4 days to complete
²Behaviour *Rotarod: 300 mice take 2 days to complete
³Blood collection
*Cytokine: 300 mice take 5 days to complete
*CBC: 300 mice take 5 days to complete *FACS: 300 mice take 5 days to complete *Nightingale: 300 mice take 5 days to complete
⁴Hemodynamics:
*blood pressure: 300 mice take 5 days to complete
⁵Urine: *urine collection: 300 mice take 5 days to complete

### Example 4. Trial of Dietary Supplements for Improving Frailty in a Human

Patient Selection

A random population of adult individuals above the age of 40 and with no signs of terminal or mental illness is subjected to analysis using the 70-item CSHA Frailty Index (Theou et al. Age Ageing 42: 614-619 (2013)) as described previously herein. A subset may also be assessed for biomarkers of aging and physiologic markers of age-related chronic disease states.

Individuals with a score suggesting that they are pre-frail are then evaluated for secondary measures: individuals with uncontrolled diabetes mellitus, cognitive impairment, malnourishment, or who regularly take alpha-ketoglutarate supplements are excluded.

Individuals not excluded by the secondary evaluation are then randomized into two groups (experimental group receiving a composition of the active agent and control group receiving placebo) using block randomization and stratification by sex. Numbers are assigned to subjects and randomized selection of the numbers is performed by a research team member uninvolved in the study. Subjects are directed to take the active agents or placebo according to the schedule established.

Data Collection

Individuals selected are subjected to a baseline assessment at the start of the study, including detailed sociodemographic data, medical history, a frailty status assessment, evaluation of physical function, evaluation of cognitive function, assessment of nutritional status, and evaluation of selected frailty biomarkers via blood sampling.

A second assessment is performed at the midpoint of the study, including a frailty status assessment, evaluation of physical function, evaluation of cognitive function, and assessment of nutritional status.

A third assessment is performed at the end of the study, including the same assessments as at baseline. All assessments are performed by medical professionals blinded to patient assignment to treatment or control groups.

At the end of the study, appropriate statistical methods (for e.g. Mann-Whitney test was employed for the continuous variables and chi-square test for the categorical variables) are applied to determine the characteristic differences between the two groups. Differences in frailty index and frailty biomarkers are evaluated to determine the effect of administration of active agents to pre-frail humans.

### Example 5. Trial of Active Agents Dietary Supplements

The study is an open label safety study, with no placebo or blinding and no randomization. Volunteers take a composition comprising an active agent over a 9-month period in the form of a tablet. The volunteers are divided into three cohorts. The volunteers are monitored for changes in general health, for example, via responses to a questionnaire, and lab panels such as comprehensive metabolic profile, complete blood count and lipid panel, blood pressure, heart rate, cardiac output, stem cells, cells senescent, IGF1, and inflammation CRP.

### Study Type:

Safety trial

### Study Design:

Length: Nine months
Primary Purpose: Safety assessment

### Cohorts:

TABLE 3

| | | Dose (# of | |
|---|---|---|---|
| Cohort | # of Volunteers | Tablets) | Regimen |
| 1 | 10-30 | 1 | QD/evening after a meal & |
| | | | without other medication^{a} |
| 2 | 10-30 | 2 | QD/evening after a meal & |
| | | | without other medication^{a} |
| 3 | 10-30 | 3 | QD/evening after a meal & |
| | | | without other medication^{a} |
| 4 | 10-30 | 4 | QD/evening after a meal & |
| | | | without other medication^{a} |

^{a}All medications should be taken in the morning

### Primary Outcome Measures:

Safety and tolerability of single and multiple doses of a composition described herein may be measured by adverse events (AEs), physical examinations (PE), vital signs (VS), laboratory safety tests, urinalysis and 12-lead electrocardiograms (ECG).

### Secondary Outcome Measures:

Pharmacokinetics of single and multiple doses of a composition described herein may be measured by maximum observed concentration (Cmax). Time Frame: 30 days
Pharmacokinetics of single and multiple doses of a composition described herein may be measured by area under the curve (AUC). Time Frame: 30 days
Pharmacokinetics of single and multiple doses of a composition described herein may be measured by terminal half life (t½). Time Frame: 30 days
Pharmacokinetics of single and multiple doses of a composition described herein may be measured by volume of distribution (Vd). Time Frame: 30 days
Pharmacokinetics of single and multiple doses of a composition described herein may be measured by elimination rate constant (Kel). Time Frame: 30 days
Pharmacokinetics of single and multiple doses of a composition described herein may be measured by accumulation ratio. Time Frame: 30 days

### Inclusion Criteria:

Men 40-65 years
Female 40-65 years

### Exclusion Criteria:

Females of child-bearing age
Pregnant females
Lactating females
Diabetics
Heart disease
Cancer
Morbidly obese

### Exemplary Questionnaire:

How do you feel today?
Have you noticed any changes in your health?
Have you had any gastrointestinal problems?
Have you noticed any changes in hair?
Have you noticed any changes in hair color (such as darkening of the hair)?
Have you noticed any improvements in your memory, such as better short term recall?
Any improvements in your balance?
Any improvements in your vision?
Any improvements in your stamina or sense of well-being?

### Example 6. Trial of Calcium Alpha-Ketoglutarate (CaAKG) and Nicotinamide Riboside (NR)

This study is a randomized, double-blind, placebo-controlled trial in adult men ages 45-65 years and postmenopausal women to age 65 years. Participants in the Calcium Alpha-Ketoglutarate (CaAKG) and Nicotinamide Riboside (NR) Trial take the study product (tablet or capsule containing CaAKG and NR) or a placebo for up to 9 months.

### Study Type:

Efficacy and Safety

### Study Design:

Length: Nine months

Primary Objective: The primary objective is to observe the effect, if any, of CaAKG and NR products on the blood analytes or plasma metabolites
Secondary Objective: The secondary objective is to determine the safety of oral administration of CaAKG and NR products.

### Tertiary/Exploratory/Correlative Objectives:

Tertiary/Exploratory objectives are: 1) amount of DNA methylation during the oral administration of study product and 2) to determine if a change in the biological age of the participants occurred during the study.

### Primary Outcome Measures:

During the study, vital signs and blood samples are collected, and the following analytes are evaluated:

a) Vital Signs Assessment:
Height (Day 1 only)
Weight
Blood Pressure
Heart rate
Temperature

b) Complete Blood Count
WBC (White Blood Cell Count)
Hemoglobin
Hematocrit
Platelet Count/L
MCV (Mean Corpuscular Volume)
MCH (Mean Corpuscular Hemoglobin)
MCHC (Mean Corpuscular Hemoglobin Concentration)
RBC (Red Blood Cell Count)
RDW-CV (Red Cell Distribution Width)
MPV (Mean Platelet Volume)
Neutrophils
Neutrophil %
Lymphocyte Count
Lymphocyte %
Monocyte Count
Monocyte %
Eosinophils
Eosinophil %
Basophils
Basophil %

c) Comprehensive Metabolic Panel
Sodium
Potassium
Chloride
Carbon Dioxide (CO2)
Urea Nitrogen (BUN)
Creatinine
Glucose
Calcium
AST (Aspartate Aminotransferase)
ALT (Alanine Aminotransferase)
Alk Phos (Alkaline Phosphatase)
Bilirubin, Total
Alkaline Phosphatase
Albumin
Protein, Total
Anion Gap
Estimated Glomerular Filtration Rate (eGFR), MDRD Equation

d) Lipid Panel
Cholesterol, Total
LDL (Low Density Lipoprotein) Cholesterol
HDL (High Density Lipoprotein) Cholesterol
Triglyceride
Cholesterol, Total

e) Additional Lab Tests
Hemoglobin A1C
Uric Acid
CRP

f) Plasma Metabolites

At each clinical site visit participants are asked to complete the following questionnaire:

1. How do you feel today? Please circle a number.
1 (poor)
2
3
4
5 (very good)

2. How would you describe your daily activity level?
1 (inactive)
2
3
4
5 (very active)

3. How would you rate your physical energy level?
1 (low)
2
3
4
5 (high)

4. How would you rate your cardiovascular endurance level?
1 (low)
2
3
4
5 (high)

5. How would you describe your ability to focus?
1 (low)
2
3
4
5 (high)

6. How would you rate your level of memory recall?
1 (poor)
2
3
4
5 (good)

7. How would you describe your quality of sleep?
1 (low)
2
3
4
5 (high)

8. How would you rate the youthfulness of your hair?
1 (poor)
2
3
4
5 (good)
N/A

9. How many minutes per week do you exercise including light exercise?

10. Are you taking a multi-vitamin? Circle One.
YES NO
If YES, what are you taking?

11. Are you taking a dietary supplement beside? Circle One.
YES NO
If YES, what are you taking?

12. Is there any other relevant information about the product you'd like to share?

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Embodiments

Embodiment 1 provides a method of delaying onset or delaying progression of frailty in a subject in need thereof comprising administering to the subject a therapeutically effective amount of nicotinamide riboside (NR) and α-ketoglutarate (AKG).

Embodiment 2 provides the method of embodiment 1, wherein AKG is formulated as a calcium salt of AKG.

Embodiment 3 provides the method of any one of embodiments 1-2, wherein NR and AKG is administered to the subject as a single composition.

Embodiment 4 provides the method of any one of embodiments 1-2, wherein NR and AKG is administered to the subject as separate compositions.

Embodiment 5 provides the method of embodiment 4, wherein NR and AKG is administered to the subject in a 24-hour period.

Embodiment 6 provides the method of any one of embodiments 1-5, wherein pterostilbene is administered to the subject.

Embodiment 7 provides the method of any one of embodiments 1-6, wherein the delaying onset or delaying progression of frailty comprises delaying onset or delaying progression of a frailty phenotype.

Embodiment 8 provides the method of embodiment 7, wherein the frailty phenotype is dermatitis.

Embodiment 9 provides the method of embodiment 7, wherein the frailty phenotype is kyphosis.

Embodiment 10 provides the method of embodiment 7, wherein the frailty phenotype is tremor.

Embodiment 11 provides the method of embodiment 7, wherein the frailty phenotype is alopecia.

Embodiment 12 provides the method of any one of embodiments 1-11, wherein the subject is a mammal.

Embodiment 13 provides the method of embodiment 12, wherein the mammal is a human.

Embodiment 14 provides the method of embodiment 12, wherein the mammal is a dog.

Embodiment 15 provides the method of embodiment 12, wherein the mammal is a cat.

Embodiment 16 provides the method of embodiment 12, wherein the mammal is livestock.

Embodiment 17 provides the method of embodiment 16, wherein the livestock is selected from the group consisting of cattle, sheep, goats, swine, poultry, and equine animals.

Embodiment 18 provides the method of any one of embodiments 1-17, wherein the NR and AKG are administered once daily.

Embodiment 19 provides the method of any one of embodiments 1-17, wherein the NR and AKG are administered twice daily.

Embodiment 20 provides the method of any one of embodiments 1-17, wherein the NR and AKG are administered in the morning and evening.

Embodiment 21 provides the method of any one of embodiments 1-17, wherein the NR and AKG are administered once a week.

Embodiment 22 provides the method of any one of embodiments 1-17, wherein the NR and AKG are administered once a month.

Embodiment 23 provides the method of any one of embodiments 1-22, wherein the NR and AKG are formulated into an orally administered form.

Embodiment 24 provides the method of embodiment 23, wherein the orally administered form comprises a tablet, a powder, a suspension, a serum, an emulsion, a capsule, a granule, a pill, a gel, a solution, or a syrup.

Embodiment 25 provides the method of embodiment 23, wherein the orally administered form is a sustained release dosage form.

Embodiment 26 provides the method of embodiment 23, wherein the orally administered form is formulated into animal feed.

Embodiment 27 provides the method of embodiment 23, wherein the orally administered form is the gel.

Embodiment 28 provides the method of any one of embodiments 1-23, wherein the NR and AKG are formulated into a topically administered form.

Embodiment 29 provides the method of embodiment 28, wherein the topically administered form is a cream, a foam, a gel, a lotion, an ointment, or a serum.

Embodiment 30 provides A method for extending healthspan in a subject in need thereof comprising administering to the subject a therapeutically effective amount of a nicotinamide riboside (NR) and α-ketoglutarate (AKG).

Embodiment 31 provides the method of embodiment 30, wherein AKG is formulated as a calcium salt of AKG.

Embodiment 32 provides the method of embodiment 30 or 31, wherein NR and AKG is administered to the subject as a single composition.

Embodiment 33 provides the method of embodiment 30 or 31, wherein NR and AKG is administered to the subject as separate compositions.

Embodiment 34 provides the method of embodiment 33, wherein NR and AKG is administered to the subject in a 24-hour period.

Embodiment 35 provides the method of any one of embodiments 30-34, wherein pterostilbene is administered to the subject.

Embodiment 36 provides the method of any one of embodiments 30-35, wherein extending healthspan comprises a delay in onset or progression of an age-related phenotype.

Embodiment 37 provides the method of embodiment 36, wherein the age-related phenotype is selected from the group consisting of graying hair, hair loss, and increased cell senescence.

Embodiment 38 provides the method of embodiment 36, wherein the age-related phenotype is graying hair.

Embodiment 39 provides the method of embodiment 36, wherein the age-related phenotype is hair loss.

Embodiment 40 provides the method of embodiment 36, wherein the age-related phenotype is increased cell senescence.

Embodiment 41 provides the method of any one of embodiments 30-40, wherein the subject is a mammal.

Embodiment 42 provides the method of embodiment 41, wherein the mammal is a human.

Embodiment 43 provides the method of embodiment 41, wherein the mammal is a dog.

Embodiment 44 provides the method of embodiment 41, wherein the mammal is a cat.

Embodiment 45 provides the method of embodiment 41, wherein the mammal is livestock.

Embodiment 46 provides the method of embodiment 45, wherein the livestock is selected from the group consisting of cattle, sheep, goats, swine, poultry, and equine animals.

Embodiment 47 provides the method of any one of embodiments 30-46, wherein the NR and AKG are administered once daily.

Embodiment 48 provides the method of any one of embodiments 30-46, wherein the NR and AKG are administered twice daily.

Embodiment 49 provides the method of any one of embodiments 30-46, wherein the NR and AKG are administered in the morning and evening.

Embodiment 50 provides the method of any one of embodiments 30-46, wherein the NR and AKG are administered once a week.

Embodiment 51 provides the method of any one of embodiments 30-46, wherein the NR and AKG are administered once a month.

Embodiment 52 provides the method of any one of embodiments 30-51, wherein the NR and AKG are formulated into an orally administered form.

Embodiment 53 provides the method of embodiment 52, wherein the orally administered form comprises a tablet, a powder, a suspension, a serum, an emulsion, a capsule, a granule, a pill, a gel, a solution, or a syrup.

Embodiment 54 provides the method of embodiment 52, wherein the orally administered form is a sustained release dosage form.

Embodiment 55 provides the method of embodiment 52, wherein the orally administered form is formulated into animal feed.

Embodiment 56 provides the method of embodiment 52, wherein the orally administered form is the gel.

Embodiment 57 provides the method of any one of embodiments 30-51, wherein the NR and AKG are formulated into a topically administered form.

Embodiment 58 provides the method of embodiment 57, wherein the topically administered form is a cream, a foam, a gel, a lotion, an ointment, or a serum.

Embodiment 59 provides A method for compressing morbidity in a subject in need thereof comprising administering to the subject a therapeutically effective amount of a nicotinamide riboside (NR) and α-ketoglutarate (AKG).

Embodiment 60 provides the method of embodiment 59, wherein AKG is formulated as a calcium salt of AKG.

Embodiment 61 provides the method of embodiment 59 or 60, wherein NR and AKG is administered to the subject as a single composition.

Embodiment 62 provides the method of embodiment 59 or 60, wherein NR and AKG is administered to the subject as separate compositions.

Embodiment 63 provides the method of embodiment 62, wherein NR and AKG is administered to the subject in a 24-hour period.

Embodiment 64 provides the method of any one of embodiments 59-63, wherein pterostilbene is administered to the subject.

Embodiment 65 provides the method of any one of embodiments 59-64, wherein compressing morbidity comprises a shortening of the time between appearance of a marker of morbidity and end of life.

Embodiment 66 provides the method of embodiment 65, wherein the marker of morbidity comprises a first heart attack, a first dyspnea from emphysema, a first disability from osteoporosis, or a first memory loss of a certain magnitude.

Embodiment 67 provides the method of any one of embodiments 59-66, wherein the subject is a mammal.

Embodiment 68 provides the method of embodiment 67, wherein the mammal is a human.

Embodiment 69 provides the method of embodiment 67, wherein the mammal is a dog.

Embodiment 70 provides the method of embodiment 67, wherein the mammal is a cat.

Embodiment 71 provides the method of embodiment 67, wherein the mammal is livestock.

Embodiment 72 provides the method of embodiment 71, wherein the livestock is selected from the group consisting of cattle, sheep, goats, swine, poultry, and equine animals.

Embodiment 73 provides the method of any one of embodiments 59-72, wherein the NR and AKG are administered once daily.

Embodiment 74 provides the method of any one of embodiments 59-72, wherein the NR and AKG are administered twice daily.

Embodiment 75 provides the method of any one of embodiments 59-72, wherein the NR and AKG are administered in the morning and evening.

Embodiment 76 provides the method of any one of embodiments 59-72, wherein the NR and AKG are administered once a week.

Embodiment 77 provides the method of any one of embodiments 59-72, wherein the NR and AKG are administered once a month.

Embodiment 78 provides the method of any one of embodiments 59-77, wherein the NR and AKG are formulated into an orally administered form.

Embodiment 79 provides the method of embodiment 78, wherein the orally administered form comprises a tablet, a powder, a suspension, a serum, an emulsion, a capsule, a granule, a pill, a gel, a solution, or a syrup.

Embodiment 80 provides the method of embodiment 78, wherein the orally administered form is a sustained release dosage form.

Embodiment 81 provides the method of embodiment 78, wherein the orally administered form is formulated into animal feed.

Embodiment 82 provides the method of embodiment 79, wherein the orally administered form is the gel.

Embodiment 83 provides the method of any one of embodiments 59-77, wherein the NR and AKG are formulated into a topically administered form.

Embodiment 84 provides the method of embodiment 83, wherein the topically administered form is a cream, a foam, a gel, a lotion, an ointment, or a serum.

Embodiment 85 provides A method for helping to maintain health in a subject in need thereof comprising administering to the subject a therapeutically effective amount of a nicotinamide riboside (NR) and α-ketoglutarate (AKG).

Embodiment 86 provides Embodiment 2 provides the method of embodiment 85, wherein AKG is formulated as a calcium salt of AKG.

Embodiment 87 provides the method of embodiment 85 or 86, wherein NR and AKG is administered to the subject as a single composition.

Embodiment 88 provides the method of embodiment 85 or 86, wherein NR and AKG is administered to the subject as separate compositions.

Embodiment 89 provides the method of embodiment 88, wherein NR and AKG is administered to the subject in a 24-hour period.

Embodiment 90 provides the method of any one of embodiments 85-89, wherein pterostilbene is administered to the subject.

Embodiment 91 provides the method of any one of embodiments 85-90, wherein helping to maintain health comprises helping to maintain a healthy musculoskeletal system.

Embodiment 92 provides the method of embodiment 91, wherein helping to maintain a healthy musculoskeletal system comprises helping to maintain grip strength.

Embodiment 93 provides the method of embodiment 91, wherein helping to maintain a healthy musculoskeletal system comprises helping to maintain normal spine curvature.

Embodiment 94 provides the method of embodiment 91, wherein helping to maintain a healthy musculoskeletal system comprises helping to maintain a normal gait.

Embodiment 95 provides the method of embodiment 91, wherein helping to maintain a healthy musculoskeletal system comprises helping to maintain normal muscle mass.

Embodiment 96 provides the method of any one of embodiments 85-90, wherein helping to maintain health comprises helping to maintain a healthy auditory system.

Embodiment 97 provides the method of any one of embodiments 85-90, wherein helping to maintain health comprises helping to maintain a healthy ocular system.

Embodiment 98 provides the method of embodiment 97, wherein helping to maintain a healthy ocular system comprises helping to maintain normal vision.

Embodiment 99 provides the method of any one of embodiments 85-90, wherein helping to maintain health comprises helping to maintain a healthy cardiovascular system.

Embodiment 100 provides the method of any one of embodiments 85-90, wherein helping to maintain health comprises helping to maintain a healthy respiratory system.

Embodiment 101 provides the method of any one of embodiments 85-90, wherein helping to maintain health comprises helping to maintain a healthy metabolism.

Embodiment 102 provides the method of embodiment 101, wherein helping to maintain a healthy metabolism comprises helping to maintain a normal body temperature.

Embodiment 103 provides the method of embodiment 101, wherein helping to maintain a healthy metabolism comprises helping to maintain a healthy body weight.

Embodiment 104 provides the method of any one of embodiments 85-103, wherein the subject is a mammal.

Embodiment 105 provides the method of embodiment 104, wherein the mammal is a human.

Embodiment 106 provides the method of embodiment 104, wherein the mammal is a dog.

Embodiment 107 provides the method of embodiment 104, wherein the mammal is a cat.

Embodiment 108 provides the method of embodiment 104, wherein the mammal is livestock.

Embodiment 109 provides the method of embodiment 108, wherein the livestock is selected from the group consisting of cattle, sheep, goats, swine, poultry, and equine animals.

Embodiment 110 provides the method of any one of embodiments 85-109, wherein the NR and AKG are administered once daily.

Embodiment 111 provides the method of any one of embodiments 85-109, wherein the NR and AKG are administered twice daily.

Embodiment 112 provides the method of any one of embodiments 85-109, wherein the NR and AKG are administered in the morning and evening.

Embodiment 113 provides the method of any one of embodiments 85-109, wherein the NR and AKG are administered once a week.

Embodiment 114 provides the method of any one of embodiments 85-109, wherein the NR and AKG are administered once a month.

Embodiment 115 provides the method of any one of embodiments 85-114, wherein the NR and AKG are formulated into an orally administered form.

Embodiment 116 provides the method of embodiment 115, wherein the orally administered form comprises a tablet, a powder, a suspension, a serum, an emulsion, a capsule, a granule, a pill, a gel, a solution, or a syrup.

Embodiment 117 provides the method of embodiment 115, wherein the orally administered form is a sustained release dosage form.

Embodiment 118 provides the method of embodiment 115, wherein the orally administered form is formulated into animal feed.

Embodiment 119 provides the method of embodiment 115, wherein the orally administered form is the gel.

Embodiment 120 provides the method of any one of embodiments 85-114, wherein the NR and AKG are formulated into a topically administered form.

Embodiment 121 provides the method of embodiment 120, wherein the topically administered form is a cream, a foam, a gel, a lotion, an ointment, or a serum.

Embodiment 122 provides A method for maintain hair density in a subject in need thereof comprising administering to the subject a therapeutically effective amount of a nicotinamide riboside (NR) and α-ketoglutarate (AKG).

Embodiment 123 provides the method of embodiment 122, wherein AKG is formulated as a calcium salt of AKG.

Embodiment 124 provides the method of embodiment 122 or 123, wherein NR and AKG is administered to the subject as a single composition.

Embodiment 125 provides the method of embodiment 122 or 123, wherein NR and AKG is administered to the subject as separate compositions.

Embodiment 126 provides the method of embodiment 125, wherein NR and AKG is administered to the subject in a 24-hour period.

Embodiment 127 provides the method of any one of embodiments 122-126, wherein pterostilbene is administered to the subject.

Embodiment 128 provides the method of any one of embodiments 122-127, wherein maintaining hair density comprises maintaining a healthy scalp.

Embodiment 129 provides the method of embodiment 128, wherein maintaining hair density comprises maintaining healthy hair follicles.

Embodiment 130 provides the method of embodiment 128, wherein maintaining hair density comprises maintaining healthy hair shafts.

Embodiment 131 provides the method of embodiment 128, wherein maintaining hair density comprises maintaining healthy hair bulbs.

Embodiment 132 provides the method of any one of embodiments 122-131, wherein the subject is a mammal.

Embodiment 133 provides the method of embodiment 132, wherein the mammal is a human.

Embodiment 134 provides the method of embodiment 132, wherein the mammal is a dog.

Embodiment 135 provides the method of embodiment 132, wherein the mammal is a cat.

Embodiment 136 provides the method of embodiment 132, wherein the mammal is livestock.

Embodiment 137 provides the method of embodiment 136, wherein the livestock is selected from the group consisting of cattle, sheep, goats, swine, poultry, and equine animals.

Embodiment 138 provides the method of any one of embodiments 122-137, wherein the NR and AKG are administered once daily.

Embodiment 139 provides the method of any one of embodiments 122-137, wherein the NR and AKG are administered twice daily.

Embodiment 140 provides the method of any one of embodiments 122-137, wherein the NR and AKG are administered in the morning and evening.

Embodiment 141 provides the method of any one of embodiments 122-137, wherein the NR and AKG are administered once a week.

Embodiment 142 provides the method of any one of embodiments 122-137, wherein the NR and AKG are administered once a month.

Embodiment 143 provides the method of any one of embodiments 122-142, wherein the NR and AKG are formulated into an orally administered form.

Embodiment 144 provides the method of embodiment 143, wherein the orally administered form comprises a tablet, a powder, a suspension, a serum, an emulsion, a capsule, a granule, a pill, a gel, a solution, or a syrup.

Embodiment 145 provides the method of embodiment 143, wherein the orally administered form is a sustained release dosage form.

Embodiment 146 provides the method of embodiment 143, wherein the orally administered form is formulated into animal feed.

Embodiment 147 provides the method of embodiment 224, wherein the orally administered form is the gel.

Embodiment 148 provides the method of any one of embodiments 122-142, wherein the NR and AKG are formulated into a topically administered form.

Embodiment 149 provides the method of embodiment 148, wherein the topically administered form is a cream, a foam, a gel, a lotion, an ointment, a serum, a shampoo, or a conditioner.

Embodiment 150 provides A method for maintaining hair pigmentation in a subject in need thereof comprising administering to the subject a therapeutically effective amount of a nicotinamide riboside (NR) and α-ketoglutarate (AKG).

Embodiment 151 provides the method of embodiment 150, wherein AKG is formulated as a calcium salt of AKG.

Embodiment 152 provides the method of embodiment 150 or 151, wherein NR and AKG is administered to the subject as a single composition.

Embodiment 153 provides the method of any one of embodiments 150-151, wherein NR and AKG is administered to the subject as separate compositions.

Embodiment 154 provides the method of embodiment 153, wherein NR and AKG is administered to the subject in a 24-hour period.

Embodiment 155 provides the method of any one of embodiments 150-154, wherein pterostilbene is administered to the subject.

Embodiment 156 provides the method of any one of embodiments 150-155, wherein maintaining hair pigmentation comprises helping to maintain a normal level melanin

Embodiment 157 provides the method of embodiment 150-156, wherein the subject has low vitamin B12 levels.

Embodiment 158 provides the method of embodiment 150-157, wherein maintaining hair pigmentation comprises maintaining a normal level of melanocyte stem cells.

Embodiment 159 provides the method of any one of embodiments 150-158, wherein the therapeutically effective amount of a nicotinamide riboside (NR) and α-ketoglutarate (AKG) is applied to the scalp of the subject.

Embodiment 160 provides the method of any one of embodiments 150-159, wherein the subject is a mammal.

Embodiment 161 provides the method of embodiment 160, wherein the mammal is a human.

Embodiment 162 provides the method of embodiment 160, wherein the mammal is a dog.

Embodiment 163 provides the method of embodiment 160, wherein the mammal is a cat.

Embodiment 164 provides the method of embodiment 160, wherein the mammal is livestock.

Embodiment 165 provides the method of embodiment 164, wherein the livestock is selected from the group consisting of cattle, sheep, goats, swine, poultry, and equine animals.

Embodiment 166 provides the method of any one of embodiments 150-165, wherein the NR and AKG are administered once daily.

Embodiment 167 provides the method of any one of embodiments 150-165, wherein the NR and AKG are administered twice daily.

Embodiment 168 provides the method of any one of embodiments 150-165, wherein the NR and AKG are administered in the morning and evening.

Embodiment 169 provides the method of any one of embodiments 150-165, wherein the NR and AKG are administered once a week.

Embodiment 170 provides the method of any one of embodiments 150-165, wherein the NR and AKG are administered once a month.

Embodiment 171 provides the method of any one of embodiments 150-170, wherein the NR and AKG are formulated into an orally administered form.

Embodiment 172 provides the method of embodiment 171, wherein the orally administered form comprises a tablet, a powder, a suspension, a serum, an emulsion, a capsule, a granule, a pill, a gel, a solution, or a syrup.

Embodiment 173 provides the method of embodiment 171, wherein the orally administered form is a sustained release dosage form.

Embodiment 174 provides the method of embodiment 171, wherein the orally administered form is formulated into animal feed.

Embodiment 175 provides the method of embodiment 172, wherein the orally administered form is the gel.

Embodiment 176 provides the method of any one of embodiments 150-170, wherein the NR and AKG are formulated into a topically administered form.

Embodiment 177 provides the method of embodiment 176, wherein the topically administered form is a cream, a foam, a gel, a lotion, an ointment, a serum, a shampoo, or a conditioner.

Embodiment 178 provides A method for re-growing hair in a subject in need thereof comprising administering to the subject a therapeutically effective amount of a nicotinamide riboside (NR) and α-ketoglutarate (AKG).

Embodiment 179 provides the method of embodiment 178, wherein AKG is formulated as a calcium salt of AKG.

Embodiment 180 provides the method of embodiment 178 or 179, wherein NR and AKG is administered to the subject as a single composition.

Embodiment 181 provides the method of embodiment 178 or 179, wherein NR and AKG is administered to the subject as separate compositions.

Embodiment 182 provides the method of embodiment 181, wherein NR and AKG is administered to the subject in a 24-hour period.

Embodiment 183 provides the method of any one of embodiments 178-182, wherein pterostilbene is administered to the subject.

Embodiment 184 provides The method of any one of embodiments 178-183, wherein the subject has male or female pattern baldness, alopecia areata, telogen effluvium, anagen effluvium, alopecial totalis, alopecia universalis, alopecia barbae, alopecia mucinosa, alopecia traction alopecia, scarring alopecia or trichotillomania.

Embodiment 185 provides the method of embodiment 184, wherein NR and AKG is applied to the scalp of the subject.

Embodiment 186 provides the method of any one of embodiments 178-185, wherein the subject is a mammal.

Embodiment 187 provides the method of embodiment 186, wherein the mammal is a human.

Embodiment 188 provides the method of embodiment 186, wherein the mammal is a dog.

Embodiment 189 provides the method of embodiment 186, wherein the mammal is a cat.

Embodiment 190 provides the method of embodiment 186, wherein the mammal is livestock.

Embodiment 191 provides the method of embodiment 190, wherein the livestock is selected from the group consisting of cattle, sheep, goats, swine, poultry, and equine animals.

Embodiment 192 provides the method of any one of embodiments 178-191, wherein the NR and AKG are administered once daily.

Embodiment 193 provides the method of any one of embodiments 178-191, wherein the NR and AKG are administered twice daily.

Embodiment 194 provides the method of any one of embodiments 178-191, wherein the NR and AKG are administered in the morning and evening.

Embodiment 195 provides the method of any one of embodiments 178-191, wherein the NR and AKG are administered once a week.

Embodiment 196 provides the method of any one of embodiments 178-191, wherein the NR and AKG are administered once a month.

Embodiment 197 provides the method of any one of embodiments 178-196, wherein the NR and AKG are formulated into an orally administered form.

Embodiment 198 provides the method of embodiment 197, wherein the orally administered form comprises a tablet, a powder, a suspension, a serum, an emulsion, a capsule, a granule, a pill, a gel, a solution, or a syrup.

Embodiment 199 provides the method of embodiment 197, wherein the orally administered form is a sustained release dosage form.

Embodiment 200 provides the method of embodiment 197, wherein the orally administered form is formulated into animal feed.

Embodiment 201 provides the method of embodiment 197, wherein the orally administered form is the gel.

Embodiment 202 provides the method of any one of embodiments 178-196, wherein the NR and AKG are formulated into a topically administered form.

Embodiment 203 provides the method of embodiment 286, wherein the topically administered form is a cream, a foam, a gel, a lotion, an ointment, a serum, a shampoo, or a conditioner.

What is claimed is:

1. A method of delaying onset or delaying progression of frailty in a subject in need thereof comprising administering to the subject a therapeutically effective amount of nicotinamide riboside (NR) and α-ketoglutarate (AKG).

2. The method of claim 1, wherein AKG is formulated as a calcium salt of AKG.

3. The method of claim 1, wherein NR and AKG is administered to the subject as a single composition.

4. The method of claim 1, wherein NR and AKG is administered to the subject as separate compositions.

5. The method of claim 1, wherein NR and AKG is administered to the subject in a 24 hour period.

6. The method of claim 1, wherein pterostilbene is administered to the subject.

7. The method of claim 1, wherein the delaying onset or delaying progression of frailty comprises delaying onset or delaying progression of a frailty phenotype.

8. The method of claim 7, wherein the frailty phenotype is dermatitis.

9. The method of claim 7, wherein the frailty phenotype is kyphosis.

10. The method of claim 7, wherein the frailty phenotype is tremor.

11. The method of claim 7, wherein the frailty phenotype is alopecia.

12. The method of claim 1, wherein the subject is a mammal.

13. The method of claim 12, wherein the mammal is a human.

14. The method of claim 12, wherein the mammal is a dog.

15. The method of claim 12, wherein the mammal is a cat.

16. The method of claim 12, wherein the mammal is livestock.

17. The method of claim 16, wherein the livestock is selected from the group consisting of cattle, sheep, goats, swine, poultry, and equine animals.

18. The method of claim 1, wherein the NR and AKG are administered once daily.

19. The method of claim 1, wherein the NR and AKG are administered twice daily.

20. The method of claim 1, wherein the NR and AKG are administered in the morning and evening.

21. The method of claim 1, wherein the NR and AKG are administered once a week.

22. The method of claim 1, wherein the NR and AKG are administered once a month.

23. The method of claim 1, wherein the NR and AKG are formulated into an orally administered form.

24. The method of claim 23, wherein the orally administered form comprises a tablet, a powder, a suspension, a serum, an emulsion, a capsule, a granule, a pill, a gel, a solution, or a syrup.

25. The method of claim 23, wherein the orally administered form is a sustained release dosage form.

26. The method of claim 23, wherein the orally administered form is formulated into animal feed.

27. The method of claim 23, wherein the orally administered form is the gel.

28. The method of claim 1, wherein the NR and AKG are formulated into a topically administered form.

29. The method of claim 28, wherein the topically administered form is a cream, a foam, a gel, a lotion, an ointment, or a serum.

30. A method for extending healthspan in a subject in need thereof comprising administering to the subject a therapeutically effective amount of a nicotinamide riboside (NR) and α-ketoglutarate (AKG).

31. The method of claim 30, wherein AKG is formulated as a calcium salt of AKG.

32. The method of claim 30, wherein NR and AKG is administered to the subject as a single composition.

33. The method of claim 30, wherein NR and AKG is administered to the subject as separate compositions.

34. The method of claim 33, wherein NR and AKG is administered to the subject in a 24 hour period.

35. The method of claim 30, wherein pterostilbene is administered to the subject.

36. The method of claim 30, wherein extending healthspan comprises a delay in onset or progression of an age-related phenotype.

37. The method of claim 36, wherein the age-related phenotype is selected from the group consisting of graying hair, hair loss, and increased cell senescence.

38. The method of claim 36, wherein the age-related phenotype is graying hair.

39. The method of claim 36, wherein the age-related phenotype is hair loss.

40. The method of claim 36, wherein the age-related phenotype is increased cell senescence.

41. The method of claim 30, wherein the subject is a mammal.

42. The method of claim 41, wherein the mammal is a human.

43. The method of claim 41, wherein the mammal is a dog.

44. The method of claim 41, wherein the mammal is a cat.

45. The method of claim 41, wherein the mammal is livestock.

46. The method of claim 45, wherein the livestock is selected from the group consisting of cattle, sheep, goats, swine, poultry, and equine animals.

47. The method of claim 30, wherein the NR and AKG are administered once daily.

48. The method of claim 30, wherein the NR and AKG are administered twice daily.

49. The method of claim 30, wherein the NR and AKG are administered in the morning and evening.

50. The method of claim 30, wherein the NR and AKG are administered once a week.

51. The method of claim 30, wherein the NR and AKG are administered once a month.

52. The method of claim 30, wherein the NR and AKG are formulated into an orally administered form.

53. The method of claim 52, wherein the orally administered form comprises a tablet, a powder, a suspension, a serum, an emulsion, a capsule, a granule, a pill, a gel, a solution, or a syrup.

54. The method of claim 52, wherein the orally administered form is a sustained release dosage form.

55. The method of claim 52, wherein the orally administered form is formulated into animal feed.

56. The method of claim 52, wherein the orally administered form is the gel.

57. The method of claim 30, wherein the NR and AKG are formulated into a topically administered form.

58. The method of claim 57, wherein the topically administered form is a cream, a foam, a gel, a lotion, an ointment, or a serum.
